(19) United States
(12) Patent Application Publication
(10) Pub. No.: US 2014/0348818 A1
(43) Pub. Date: Nov. 27, 2014

(54) BIOMARKERS OF KAWASAKI DISEASE

(71) Applicant: Baylor Research Institute, Dallas, TX (US)

(72) Inventors: Virginia M. Pascual, Dallas, TX (US); Zhaohui Xu, Allen, TX (US); Octavio Ramilo, Columbus, OH (US); Rolando Cimaz, Firenze (IT)

(21) Appl. No.: 14/362,857

(22) PCT Filed: Dec. 21, 2012
(86) PCT No.: PCT/US2012/071360

§ 371 Date: Jun. 4, 2014

Related U.S. Application Data
(60) Provisional application No. 61/581,199, filed on Dec. 29, 2011.

(51) Int. Cl. C12Q 1/68; G06F 19/20; A61K 31/56; G01N 33/74; C07K 16/00; A61K 31/616

(52) U.S. Cl. CPC C12Q 1/6888; C07K 16/00; A61K 31/616; A61K 31/56; G01N 33/74; G06F 19/20; A61K 2039/505
USPC 424/130.1; 435/6.12; 514/165; 514/169; 506/9; 436/501; 435/7.1; 435/7.92; 702/19

(57) ABSTRACT

Biomarkers of Kawasaki Disease (KD) are provided. In certain aspects, methods are provided for detecting KD biomarkers, such as elevated PDGFC expression. Likewise, methods of treating a subject having a biomarker of KD are described.

The application claims priority to U.S. Provisional Patent Application 61/581,199 filed on Dec. 29, 2011, hereby incorporated by reference.

### BACKGROUND OF THE INVENTION

1. Field of the Invention

The present invention relates generally to the field of medicine and medical diagnostics. More particularly, it concerns methods for detecting and treating Kawasaki disease.

2. Description of Related Art

Kawasaki disease (KD) has an age-specific distribution with most cases occurring in children between 6 months to 4 years. It is more prevalent in Japan and in children of Japanese ancestry, with an annual incidence of ˜112 cases per 100 000 children less than 5 years of age. In the United States, the incidence of Kawasaki disease has been best estimated as 4248 hospitalizations associated with Kawasaki disease in 2000, with a median age of 2 years. KD typically begins with high fevers for at least 5 days, and presents with other principal features and laboratory/clinical findings. Although the coronary arteries virtually always are involved in autopsy cases, Kawasaki disease is a generalized systemic vasculitis involving blood vessels throughout the body. Aneurysms may occur in other extraparenchymal muscular arteries such as the celiac, mesenteric, femoral, iliac, renal, axillary, and brachial arteries.

The etiology of Kawasaki disease remains unknown, although clinical and epidemiological features strongly suggest an infectious cause. An influx of neutrophils is found in the early stages (7-9 days after onset), with a rapid transition to large mononuclear cells in concert with lymphocytes (predominantly CD8⁺ T cells) and IgA plasma cells. Destruction of the internal elastic lamina and eventually fibroblastic proliferation occur at this stage and circulating levels of IL-1 and TNF-alpha are also elevated in KD patients. Active inflammation is replaced over several weeks to months by progressive fibrosis, with scar formation. However, despite detailed study, diagnosis of KD is still based on clinical symptoms and accordingly correct diagnosis often cannot be made in the early stages of disease when application of therapy could be most effective.

### SUMMARY OF THE INVENTION

In a first embodiment, there is provided a method for detecting a biomarker of KD in a subject comprising determining a EPSTI1, OASL, CEBPA, C9orf167, FHOD1, ALDH3B1, LRSAM1, SIGLEC7, SLC24A4, GAA, RRBP1, DAB2, HIST2H3C, LGALS9, GPR177, CMTM4, FBXO30, WSB2, PAPSS1, SERPINB2, ACTA2, LOC729417, ABCD1, GNB4, MITF, C1QC, CCDC24, PGM5, LOC729816, PDGFC or OLFM4 expression level a biological sample from a subject suspected of having or at risk for having KD, wherein elevated expression relative to a reference level identifies the subject as having a biomarker of KD.

In a further embodiment, there is provided a method for detecting a biomarker of KD in a subject comprising determining a LOC641518, C21orf57, UBB, FBXO7, LOC731777, BTF3, C13orf15, SFRS2B, HEMGN, HPS1, IFT52, FAM10A7, IFT52, LOC441714, IMMP2L, TMEM57, IFRD2, LOC646784, PYROXD1, MIR155HG, ZNF138, TCC39B, OR7E156P, FANCD2, XPOT, AZIN1, BLOC152, CDK2, MYLS, HRASLS2 or TMCC1 expression level a biological sample from a subject suspected of having or at risk for having KD, wherein decreased expression relative to a reference level identifies the subject as having a biomarker of KD.

In still a further embodiment, there is provided a method for detecting a biomarker of Kawasaki disease (KD) in a subject comprising determining a PDGFC expression level a biological sample from a subject suspected of having or at risk for having KD, wherein elevated PDGFC expression relative to a reference level identifies the subject as having a biomarker of KD.

In yet a further embodiment a method for treating a subject with KD is provided comprising (a) evaluating expression of a biomarker in the subject and (b) administering an anti-KD therapy to the subject if the subject comprises a KD biomarker. For example, in some aspects, evaluating expression of a biomarker can comprise measuring biomarker expression in a sample from the subject. In further aspects, evaluating expression of a biomarker can comprise analysis of a report providing a level of biomarker expression in a sample from the subject. Thus, in some aspects, a method for treating a subject with KD is provided comprising (a) evaluating expression of PDGFC in the subject and (b) administering an anti-KD therapy to the subject if the subject exhibits elevated PDGFC expression relative to a reference level.

In a further embodiment a method is provided for treating a subject with KD comprising (a) administering an anti-KD therapy to the subject; (b) evaluating expression of PDGFC in the subject; and (c) administering a further anti-KD therapy to the subject if the subject exhibits elevated PDGFC expression relative to a reference level. Thus, in certain aspects, a method of the embodiments can be defined as a method for monitoring or determining the effectiveness of an anti-KD therapy.

In still a further embodiment a method treating KD is provided comprising administering an anti-KD therapy to a subject determined to have a KD biomarker. For example, in certain aspects, a method treating KD is provided comprising administering an anti-KD therapy to a subject determined to have an elevated PDGFC expression relative to a reference level.

Certain aspects of the embodiments concern a subject suspected of having or at risk for having KD. For example, a subject can exhibit one or more of the following symptoms: oral erythema; rash; swollen lips; cracked lips; swelling of the hands; swelling of the feet; eye redness; uveitis; aseptic meningitis; lymph node inflammation; vascular inflammation; coronary aneurism; fever (e.g., a persistent fever ongoing for at least 2, 3, 4, 5 or more days); joint pain; joint swelling; or peeling skin over nail beds, palms, soles and groin area. In some aspects, the subject a is a child, such as child between the ages of 6-months and 2, 3, 4, or 5 years of age. In still further aspects, the subject is a human subject, such as a subject of Asian (e.g., Japanese) ancestry. In certain aspects, a subject can be a subject who does not comprise a KD biomarker (e.g., an elevated PDGFC expression level).

Certain aspects of embodiments concern biological samples from a subject, such as blood (e.g., serum), saliva, urine, fecal or tissue samples. In certain aspects, a sample can be obtained directly from the subject (e.g., by drawing blood from the subject). In further aspects, the sample can be a sample obtained by a third party (e.g., a doctor) or can be from a tissue or blood bank. In some aspects, samples can be processed, such as by isolating or concentrating proteins or nucleic acids (e.g., RNA) from the sample. For example, a sample can be treated to purify or partially purify proteins or nucleic acids or to removes certain proteins or nucleic acids (e.g., to remove excess globin RNA).

Aspects of the embodiments concern determining the expression a KD biomarker in a sample. For example, determining expression can comprise measuring expression of the biomarker. Expression of a biomarker can be determined by, for example, detecting RNA or protein expression or by detecting the activity of an RNA or protein. Thus, in certain aspects, determining the expression a biomarker can comprise measuring a level of expression of a RNA or protein in a sample. In further aspects, a method of the embodiments can comprise reporting (e.g., in a written or electronic report) the expression a biomarker in the sample. In still further aspects, a method of the embodiments can comprise reporting whether the sample (or the subject) has a KD biomarker.

In some embodiments, methods will involve determining or calculating a diagnostic score based on data concerning the expression level of one or more biomarker, meaning that the expression level of the one or more biomarker is at least one of the factors on which the score is based. A diagnostic score will provide information about the biological sample, such as the general probability that the sample is from a subject having KD. In certain embodiments, a probability value is expressed as a numerical integer that represents a probability of 0% likelihood to 100% likelihood that a subject has KD. In some embodiments, the probability value is expressed as a numerical integer that represents a probability of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% likelihood (or any range derivable therein) that a subject has KD.

Certain aspects of the embodiments concern determining the expression of PDGFC in a sample. For example, PDGFC RNA and/or protein expression can be determined in the sample. In certain aspects, determining expression comprises determining expression of active PDGFC (e.g., expression PDGFC RNA that encodes a functional protein). In some aspects, determining the expression of PDGFC comprises measuring a level of expression of a RNA or protein in a sample.

Methods for determining expression of a biomarker are well known in the art and any such method may be employed with respect to a KD biomarker. For example, in the case of detecting protein expression methods that can be employed include, but are not limited to, mass spectroscopy, an aptamer binding assay or an immune-detection method that employs an anti-biomarker antibody (e.g., Western blot, ELISA or IHC). In the case of determining RNA expression of a biomarker methods that can be employed include, but are not limited to, nucleic acid hybridization (e.g., Northern blot or hybridization to an array), nucleic acid sequencing or reverse transcription polymerase chain reaction (RT-PCR).

Some aspects of the embodiments comprise determining whether expression of a KD biomarker is elevated in a sample. For example, the expression of a KD biomarker (e.g., PDGFC) can be compared to a reference expression level, such as an expression level in sample from a healthy subject or a subject that does not have KD. For instance, in the case of PDGFC, an elevated level of RNA expression can comprise expression of between about 3-, 4-, 5-, 6-, 7-, 8-, 9- or 10- and about 20-, 25-, 30-, 35-, 40-, 45-, or 50-fold greater PDGFC RNA expression relative to a reference level of expression. In still further aspects, determining a PDGFC expression level can comprise determining a level of expression of an RNA encoding an active PDGFC polypeptide (e.g., an RNA encoding the sequence of SEQ ID NO:1). Thus, in certain aspects, determining PDGFC expression can comprise determining expression of a PDGFC RNA encoding an active PDGFC polypeptide or determining a ratio of expression of an RNA encoding an active PDGFC polypeptide relative to a PDGFC RNA that does not encode an active polypeptide.

Still further embodiments concern determining the expression of a KD biomarker in a sample and the expression of at least a second gene. For example, the second gene can be a control gene. In some aspects, the expression of a control gene can be used to normalize the expression level of a KD biomarker, e.g., to account for difference in sample size or sample quality. In further aspects, the second gene can be a further biomarker. For example, in certain aspects, methods of the embodiments comprise determine PDGFC expression in a sample and determining the expression of at least a second gene selected from the group consisting of LOC641518, C21orf57, UBB, FBXO7, LOC731777, BTF3, C13orf15, SFRS2B, HEMGN, HPS1, IFT52, FAM10A7, IFT52, LOC441714, IMMP2L, TMEM57, IFRD2, LOC646784, PYROXD1, MIR155HG, ZNF138, TCC39B, OR7E156P, FANCD2, XPOT, AZIN1, BLOC152, CDK2, MYL5, HRASLS2, TMCC1, EPSTI1, OASL, CEBPA, C9orf167, FHOD1, ALDH3B1, LRSAM1, SIGLEC7, SLC24A4, GAA, RRBP1, DAB2, HIST2H3C, LGALS9, GPR177, CMTM4, FBXO30, WSB2, PAPSS1, SERPINB2, ACTA2, LOC729417, ABCD1, GNB4, MITF, C1QC, CCDC24, PGM5, LOC729816 and OLFM4. In yet further aspects, expression from at least a second gene associated with KD is determined in a sample wherein the second gene is TNFα, IL-1 or one of the genes described in U.S. Patent Publn. 20110189698 or 20090304680, incorporated herein by reference. Thus, in some aspects, a method can comprise determining the expression of at least 2, 3, 4, 5, 6, 7, 8, 9, 10, 11, 12, 13, 14, 15, 16, 17, 18, 19, 20, 25, or 30 biomarkers in a sample from a subject suspected of having or at risk for having KD.

Further aspects of the embodiments concern treatment of a subject having or diagnosed with KD or a subject determined to have a biomarker of KD (e.g., a subject determined to have elevated PDGFC expression). For example, a subject can be treated with an appropriate anti-KD therapy, such as by administration of IgG, aspirin, corticosteroids and/or an anti-TNFα therapy. In still further aspects, there is provided a method of treating a subject determined not to have a biomarker of KD comprising administering an anti-inflammatory therapy that does not include IgG administration.

In still a further embodiment there is provided a tangible computer-readable medium comprising computer-readable code that, when executed by a computer, causes the computer to perform operations comprising (a) receiving information corresponding to a level of expression of a KD biomarker in a sample from a subject suspected of having or at risk for having KD; and (b) determining a relative level of expression of the KD biomarker compared to a reference level. For example, the computer-readable code that can causes the computer to perform operations comprising (a) receiving information corresponding to a level of expression of PDGFC in a sample from a subject suspected of having or at risk for having KD; and (b) determining a relative level of expression of PDGFC compared to a reference level, wherein elevated PDGFC expression relative to the reference level indicates the presence of a biomarker of KD. In certain aspects, the computer-readable code further causes the computer to receive information corresponding to a reference level of expression of a KD biomarker (e.g., PDGFC) in a sample from a healthy subject. In further aspects, the computer-readable medium comprises a reference level (e.g. a PDGFC reference level) stored in said medium.

In still further aspects, a computer-readable medium comprises code for performing one or more additional operations comprising: sending information corresponding to the relative level of expression of biomarker expression, such as PDGFC, to a tangible data storage device and/or calculating a diagnostic score for the sample, wherein the diagnostic score is indicative of the probability that the sample is from a subject having KD. In still further aspects, computer-readable medium comprises code for receiving information corresponding to a level of expression of at one of LOC641518, C21orf57, UBB, FBXO7, LOC731777, BTF3, C13orf15, SFRS2B, HEMGN, HPS1, IFT52, FAM10A7, IFT52, LOC441714, IMMP2L, TMEM57, IFRD2, LOC646784, PYROXD1, MIR155HG, ZNF138, TCC39B, OR7E156P, FANCD2, XPOT, AZIN1, BLOC152, CDK2, MYL5, HRASLS2, TMCC1, EPSTI1, OASL, CEBPA, C9orf167, FHOD1, ALDH3B1, LRSAM1, SIGLEC7, SLC24A4, GAA, RRBP1, DAB2, HIST2H3C, LGALS9, GPR177, CMTM4, FBXO30, WSB2, PAPSS1, SERPINB2, ACTA2, LOC729417, ABCD1, GNB4, MITF, C1QC, CCDC24, PGM5, LOC729816 or OLFM4 in a sample from a subject suspected of having or at risk for having KD.

A processor or processors can be used in performance of the operations driven by the example tangible computer-readable media disclosed herein. Alternatively, the processor or processors can perform those operations under hardware control, or under a combination of hardware and software control. For example, the processor may be a processor specifically configured to carry out one or more those operations, such as an application specific integrated circuit (ASIC) or a field programmable gate array (FPGA). The use of a processor or processors allows for the processing of information (e.g., data) that is not possible without the aid of a processor or processors, or at least not at the speed achievable with a processor or processors. Some embodiments of the performance of such operations may be achieved within a certain amount of time, such as an amount of time less than what it would take to perform the operations without the use of a computer system, processor, or processors, including no more than one hour, no more than 30 minutes, no more than 15 minutes, no more than 10 minutes, no more than one minute, no more than one second, and no more than every time interval in seconds between one second and one hour.

Some embodiments of the present tangible computer-readable media may be, for example, a CD-ROM, a DVD-ROM, a flash drive, a hard drive, or any other physical storage device. Some embodiments of the present methods may include recording a tangible computer-readable medium with computer-readable code that, when executed by a computer, causes the computer to perform any of the operations discussed herein, including those associated with the present tangible computer-readable media. Recording the tangible computer-readable medium may include, for example, burning data onto a CD-ROM or a DVD-ROM, or otherwise populating a physical storage device with the data. In certain aspects, a tangible computer-readable media can be included in a kit of the embodiments.

Also provided are kits containing the disclosed compositions or compositions used to implement the disclosed methods. In some embodiments, kits can be used to determine the expression one or more biomarker. In certain embodiments, a kit contains, contains at least, or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, or any range and combination derivable therein, nucleic acid probes including those that may specifically hybridize under stringent conditions to RNA biomarkers disclosed herein. In further embodiments, kits or methods may involve nucleic acid probes, which may be capable of specifically detecting RNA expression one or more of the following LOC641518, C21orf57, UBB, FBXO7, LOC731777, BTF3, C13orf15, SFRS2B, HEMGN, HPS1, IFT52, FAM10A7, IFT52, LOC441714, IMMP2L, TMEM57, IFRD2, LOC646784, PYROXD1, MIR155HG, ZNF138, TCC39B, OR7E156P, FANCD2, XPOT, AZIN1, BLOC152, CDK2, MYL5, HRASLS2, TMCC1, EPSTI1, OASL, CEBPA, C9orf167, FHOD1, ALDH3B1, LRSAM1, SIGLEC7, SLC24A4, GAA, RRBP1, DAB2, HIST2H3C, LGALS9, GPR177, CMTM4, FBXO30, WSB2, PAPSS1, SERPINB2, ACTA2, LOC729417, ABCD1, GNB4, MITF, C1QC, CCDC24, PGM5, LOC729816, PDGFC and OLFM4.

In yet a further embodiment, a kit of the embodiments comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more, or any range and combination derivable therein, antibodies that specifically binds to the biomarkers disclosed herein. In further embodiments, kits or methods may involve antibodies, which may be capable of specifically detecting protein expression one or more of the following LOC641518, C21orf57, UBB, FBXO7, LOC731777, BTF3, C13orf15, SFRS2B, HEMGN, HPS1, IFT52, FAM10A7, IFT52, LOC441714, IMMP2L, TMEM57, IFRD2, LOC646784, PYROXD1, MIR155HG, ZNF138, TCC39B, OR7E156P, FANCD2, XPOT, AZIN1, BLOC152, CDK2, MYL5, HRASLS2, TMCC1, EPSTI1, OASL, CEBPA, C9orf167, FHOD1, ALDH3B1, LRSAM1, SIGLEC7, SLC24A4, GAA, RRBP1, DAB2, HIST2H3C, LGALS9, GPR177, CMTM4, FBXO30, WSB2, PAPSS1, SERPINB2, ACTA2, LOC729417, ABCD1, GNB4, MITF, C1QC, CCDC24, PGM5, LOC729816, PDGFC and OLFM4.

In yet a further embodiment a kit may comprise at least a first nucleic acid probe that can specifically hybridize to a PDGFC RNA that encodes a functional PDGFC protein (e.g., SEQ ID NO: 1) and at least second nucleic acid probe that can specifically hybridize to a PDGFC RNA that does not encode functional PDGFC protein (e.g., SEQ ID NO: 3). For example, a kit of the embodiments can comprise at least a first primer pair that can specifically amplify a segment of sequence from a PDGFC RNA that encodes a functional PDGFC protein (e.g., SEQ ID NO: 1) and at least second primer pair that can specifically amplify a segment of sequence from a PDGFC RNA that does not encode functional PDGFC protein (e.g., SEQ ID NO: 3).

As used herein the specification, “a” or “an” may mean one or more. As used herein in the claim(s), when used in conjunction with the word “comprising”, the words “a” or “an” may mean one or more than one.

It is contemplated that any embodiment discussed herein can be implemented with respect to any disclosed method or composition, and vice versa. Any embodiment discussed with respect to a particular pancreatic disorder can be applied or implemented with respect to a different pancreatic disorder. Furthermore, the disclosed compositions and kits can be used to achieve the disclosed methods.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: A schematic representation of network of genes involved in a signaling associated with inflammation. Gene transcripts that were found to be differentially regulated in KD blood were mapped onto the signaling network. +, denotes transcripts that were up-regulated in KD. (−), denotes transcripts that were down-regulated in KD.

FIG. 2: A schematic representation of network of genes involved in a signaling associated with connective tissue development. Gene transcripts that were found to be differentially regulated in KD blood were mapped onto the signaling network. +, denotes transcripts that were up-regulated in KD. (−), denotes transcripts that were down-regulated in KD.

FIG. 3: PDGFC transcript is up-regulated 5-30 fold in the blood of KD patients. Chart shows the fold change (FC) in PDGFC transcript expression relative to the average expression for healthy controls. Results were obtained by quantitative RT-PCR in three regions of the PDGFC transcript.

FIG. 4: PDGFC transcripts encoding functional PDGFC protein are up-regulated in KD patients. Graph shows the ratio of PDGFC transcripts encoding functional protein versus transcripts that do not include a function PDGFC ORF. KD, indicates samples from KD patients. H, indicates samples from healthy subjects.

FIG. 5: PDGFC transcript levels in whole blood from KD and other febrile diseases were evaluated with quantitative RT-PCR.

FIG. 6: Microarray analysis indicates PDGFC transcription is up-regulated in KD patients.

### DESCRIPTION OF ILLUSTRATIVE EMBODIMENTS

### I. The Present Invention

Kawasaki disease is a leading cause of acquired heart disease in children, with more than 80% of KD cases presenting between the ages of 6 months and 4 years. The cause of KD is unknown and, although an infectious agent is suspected, genetics and environment also appear to play role in the disease. Currently KD diagnosis can only be achieved by a combination of clinical features and accordingly rapid diagnosis is not possible. Unfortunately, a delayed diagnosis (and resulting delays in the application of proper treatment) increase the probability of serious complications. In fact, while coronary aneurisms develop in as many as 20% of untreated patients, only 5% of treated patients develop such an aneurism. Thus, rapid methods for diagnosis of KD are in great need.

The studies detailed here examined gene expression levels of KD patients compared with other IL-1 related diseases—Neonatal onset multisystem inflammatory disease (NOMID) and Systemic Onset Juvenile Idiopathic Arthritis (sJIA). Overall the gene expression patterns in these three diseases were found to be very similar. However, a number of genes were identified that were specifically up- or down-regulated only in the case of KD. In particular, platelet-derived growth factor C (PDGFC) was found to be specifically up-regulated in Kawasaki patients, but not in NOMID and sJIA. Likewise, platelet-derived growth factor C (PDGFC) was found to be specifically up-regulated in Kawasaki patients but not in Juvenile dermatomyositis (JDM), Systemic lupus erythematosus (SLE), Rhinovirus infection, Escherichia coli infection, Methicillin-resistant Staphylococcus aureus (MRSA) infection or staphylococcus aureus (Staph) infection. Furthermore, it was found that KD patients preferentially expressed increased levels of PDGFC transcripts that encoded functional PDGFC proteins.

Thus, the studies detailed here demonstrate that increased PDGFC expression can be used as biomarker for diagnosing KD. For example, serum samples from a patient suspected of having KD can be analyzed to determine PDGFC expression. Elevated PDGFC expression levels or elevated expression of active PDGFC RNA isoforms can thus be used to determine whether a subject has KD. Such a rapid diagnosis will likewise allow for early therapeutic intervention which could significant reduce the severity of disease and decrease possibility of developing complications, such as coronary aneurism.

### II. PDGFC

PDGFC is important in tissue growth and function, and plays a role in recruiting fibroblasts associated with drug-resistant tumors. The gene was first identified by its similarity to other members PDGF/VEGF family of genes (Reigstad et al., 2005). Two different mRNA transcripts have been identified. The shorter of the two PDGFC coding RNAs encodes a functional open reading frame (ORF) for the PDGFC protein (NM_{—}016205.2, incorporated herein by reference; SEQ ID NO: 1). The longer transcript includes an alternative splice event that places the PDGFC coding region out of frame and therefore does not encode a functional PDGFC protein (NR_{—}036641.1, incorporated herein by reference; SEQ ID NO: 3).

Certain aspects of the embodiments concern determining the expression of PDGFC in a sample. In some aspects, determining the expression of PDGFC comprises determining expression of RNA encoding a functional PDGFC protein and RNA that does not encode a functional protein. However, in certain aspects, determining the expression of PDGFC comprises determining expression of RNA encoding a functional PDGFC protein or determining the expression ratio of RNA encoding a functional PDGFC protein to RNA that does not encode a function protein. For example, a subject having elevated expression of RNA encoding a functional PDGFC protein or having an increased expression ratio of RNA encoding a functional PDGFC protein to RNA that does not encode a function protein can be determined to have a biomarker of KD.

A skilled artisan will recognize that a variety of methods can be used to determine PDGFC RNA expression and will be able to discern between the expression of RNA that encodes a functional protein (e.g., SEQ ID NO: 1) and RNA that does not encode function protein (e.g., SEQ ID NO: 3). For example, hybridization probes can be employed that hybridize only to regions of sequence that are unique to one RNA or the other. Likewise, primers can be used for RT-PCR that are only able to amplify sequence from one RNA or the other, or that generate amplicons of different length in the case of the different PDGFC RNAs. One such detection method that can quantify the functional versus non-functional RNAs is exemplified herein.

### III. Detecting KD Biomarkers

Certain embodiments concern detecting, either in vivo or in a sample, expression of a KD biomarker. For example, in some embodiments, expression of a KD biomarker such as PDGFC can be detected by measuring expression or activity of the protein. In further aspects, expression of a KD biomarker can be detected by measuring expression of an RNA encoding the biomarker.

A. Nucleic Acid Detection

In some embodiments, assessing expression of a KD biomarker, such as PDGFC, can involve quantifying mRNA expression. Northern blotting techniques are well known to those of skill in the art. Northern blotting involves the use of RNA as a target. Briefly, a probe is used to target an RNA species that has been immobilized on a suitable matrix, often a filter of nitrocellulose. The different species should be spatially separated to facilitate analysis. This often is accomplished by gel electrophoresis of nucleic acid species followed by “blotting” on to the filter. Subsequently, the blotted target is incubated with a probe (such as a labeled probe) under conditions that promote denaturation and rehybridization. Because the probe is designed to base pair with the target, the probe will binding a portion of the target sequence under renaturing conditions. Unbound probe is then removed, and detection is accomplished.

In some embodiments, nucleic acids are quantified following gel separation and staining with ethidium bromide and visualization under UV light. In some embodiments, if the nucleic acid results from a synthesis or amplification using integral radio- or fluorometrically-labeled nucleotides, the products can then be exposed to x-ray film or visualized under the appropriate stimulating spectra, following separation.

In some embodiments, visualization is achieved indirectly. Following separation of nucleic acids, a labeled nucleic acid is brought into contact with the target sequence. The probe is conjugated to a chromophore or a radiolabel. In another embodiment, the probe is conjugated to a binding partner, such as an antibody or biotin, and the other member of the binding pair carries a detectable moiety. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present embodiments.

In some embodiments, reverse transcription (RT) of RNA to cDNA followed by relative quantitative PCR™ (RT-PCR™) can be used to determine the relative concentrations of specific mRNA (e.g., a PDGFC coding RNA) or even a specific mRNA species isolated from a subject (e.g., a mRNA encoding active PDGFC). By determining that the concentration of a specific mRNA or species of mRNA varies, it is shown that the gene encoding the specific mRNA species is differentially expressed. In certain aspects mRNA expression can be quantified relative to the expression of a control mRNA, such as the expression of phosphoglycerate kinase 1 (PGK1; NCBI accession no. NM_{—}000291.3, incorporated herein by reference) or TATA box binding protein (TBP; NCBI accession no. NM_{—}003194.4, incorporated herein by reference).

In some embodiments, the amplification products described above may be subjected to sequence analysis to identify specific kinds of variations using standard sequence analysis techniques. Within certain methods, exhaustive analysis of genes is carried out by sequence analysis using primer sets designed for optimal sequencing. The present embodiments provide methods by which any or all of these types of analyses may be used. Using the sequences disclosed herein, oligonucleotide primers may be designed to permit the amplification of sequences throughout a KD biomarker gene (or protein coding sequence) that may then be analyzed by direct sequencing. Likewise, DNA sequencing may be used to detect and/or quantify expression of a KD biomarker gene. Methods for such sequence include, but are not limited to, reversible terminator methods (e.g., used by Illumina® and Helicos® BioSciences), pyrosequencing (e.g., 454 sequencing from Roche) and sequencing by ligation (e.g., Life Technologies™ SOLiD™ sequencing)

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR™ products and the relative mRNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR™ for a collection of RNA populations is that the concentrations of the amplified PCR™ products must be sampled when the PCR™ reactions are in the linear portion of their curves.

The second condition that must be met for an RT-PCR™ experiment to successfully determine the relative abundances of a particular mRNA species is that relative concentrations of the amplifiable cDNAs must be normalized to some independent standard. The goal of an RT-PCR™ experiment is to determine the abundance of a particular mRNA species relative to the average abundance of all mRNA species in the sample.

Most protocols for competitive PCR™ utilize internal PCR™ standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

B. Protein Biomarker Detection

In some aspects, methods of the embodiments concern detection of the expression or activity of protein biomarkers, such as PDGFC. For example, immunodetection methods for binding, purifying, removing, quantifying and/or otherwise generally detecting protein components such as PDGFC can be employed. Antibodies prepared in accordance with the present embodiments may be employed to detect KD biomarker expression and/or KD biomarker activation. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference.

In general, the immunobinding methods include obtaining a sample suspected of containing a KD biomarker protein, polypeptide and/or peptide (e.g., PDGFC), and contacting the sample with a first anti-biomarker antibody in accordance with the present embodiments, under conditions effective to allow the formation of immunocomplexes.

These methods include methods for purifying wild type and/or mutant biomarker proteins, polypeptides and/or peptides as may be employed in purifying wild type and/or mutant biomarker proteins, polypeptides and/or peptides from patients' samples and/or for purifying recombinantly expressed wild type or mutant proteins, polypeptides and/or peptides. In these instances, the antibody removes the antigenic biomarker protein, polypeptide and/or peptide component from a sample. The antibody will preferably be linked to a solid support, such as in the form of a column matrix, and the sample suspected of containing biomarker protein antigenic component will be applied to the immobilized antibody. The unwanted components will be washed from the column, leaving the antigen immunocomplexed to the immobilized antibody, biomarker protein antigen is then collected by removing the protein and/or peptide from the column.

The immunobinding methods also include methods for detecting and quantifying the amount of a KD biomarker or activated KD biomarker in a sample. Here, one would obtain a sample suspected of containing a biomarker and contact the sample with an antibody and then detect and quantify the amount of immune complexes formed under the specific conditions.

In terms of antigen detection, the biological sample analyzed may be any sample that is suspected of containing a cell expressing a KD biomarker, such as a serum or whole blood sample, a tissue extract or another biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any KD biomarker protein antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

In some embodiments, a KD biomarker antibody (e.g., an anti-PDGFC antibody) employed in the detection may itself be linked to a detectable label, wherein one would then simply detect this label, thereby allowing the amount of the primary immune complexes in the composition to be determined. In some embodiments, the first antibody that becomes bound within the primary immune complexes may be detected by means of a second binding ligand that has binding affinity for the antibody. In certain embodiments, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR methodology. The PCR™ method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR™ reaction with suitable primers with appropriate controls. At least in theory, the enormous amplification capability and specificity of PCR™ can be utilized to detect a single antigen molecule.

The immunodetection methods of the present embodiments have evident utility in the diagnosis and prognosis of conditions such as various forms of inflammatory disease, such as KD. Here, a biological and/or clinical sample suspected of containing a KD biomarker protein, polypeptide, peptide and/or mutant is used. However, these embodiments also have applications to non-clinical samples, such as in the titering of antigen or antibody samples, for example in the identification of cellular mediators of inflammation.

In the clinical diagnosis and/or monitoring of patients with KD, the detection of biomarkers, such as increased expression or activation of PDGFC, in comparison to the levels in a corresponding biological sample (i.e., a reference level) from a normal subject is indicative of a patient with KD. However, as is known to those of skill in the art, such a clinical diagnosis would not necessarily be made on the basis of this method in isolation. Those of skill in the art are very familiar with differentiating between significant differences in types and/or amounts of biomarkers, which represent a positive identification, and/or low level and/or background changes of biomarkers. Indeed, background expression levels are often used to form a “cut-off” above which increased detection will be scored as significant and/or positive. Likewise, diagnosis can be made based upon the presence of two, three or more biomarkers and/or on the presence of a biomarker in conjunction with one or more clinical symptoms indicative of KD.

1. ELISAs

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful.

In some embodiments, the anti-biomarker antibodies of the embodiments are immobilized onto a selected surface exhibiting protein affinity, such as a well in a polystyrene microtiter plate. Then, a test composition suspected of containing the biomarker protein antigen, such as a clinical sample, is added to the wells. After binding and/or washing to remove non-specifically bound immune complexes, the bound biomarker protein antigen may be detected. Detection is generally achieved by the addition of another anti-biomarker antibody that is linked to a detectable label. This type of ELISA is a simple “sandwich ELISA”. Detection may also be achieved by the addition of a second anti-biomarker antibody, followed by the addition of a third antibody that has binding affinity for the second antibody, with the third antibody being linked to a detectable label.

In some embodiments, the samples suspected of containing the biomarker protein antigen are immobilized onto the well surface and/or then contacted with the anti-biomarker antibodies of the embodiments. After binding and/or washing to remove non-specifically bound immune complexes, the bound anti-biomarker antibodies are detected. Where the initial anti-biomarker antibodies are linked to a detectable label, the immune complexes may be detected directly. Again, the immune complexes may be detected using a second antibody that has binding affinity for the first anti-biomarker antibody, with the second antibody being linked to a detectable label.

In some embodiments, the biomarker proteins, polypeptides and/or peptides are immobilized. In some embodiments, ELISA involves the use of antibody competition in the detection. In this ELISA, labeled antibodies against a KD biomarker protein are added to the wells, allowed to bind, and/or detected by means of their label. The amount of wild type or mutant biomarker protein antigen in an unknown sample is then determined by mixing the sample with the labeled antibodies against the biomarker before and/or during incubation with coated wells. The presence of biomarker protein in the sample acts to reduce the amount of antibody against wild type or mutant protein available for binding to the well and thus reduces the ultimate signal. This is also appropriate for detecting antibodies against biomarker protein in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In some embodiments, a secondary or tertiary detection means is used rather than a direct procedure. In some such embodiments, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody may have an associated label to allow detection. In some embodiments, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween). After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

2. Immunohistochemistry

Anti-KD biomarker antibodies of the present embodiments may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections (e.g., vascular tissue sections) may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in 70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

3. Immunoelectron Microscopy

Antibodies of the present embodiments may also be used in conjunction with electron microscopy to identify intracellular tissue components. Briefly, an electron-dense label is conjugated directly or indirectly to an anti-biomarker antibody. Examples of electron-dense labels according to the embodiments are ferritin and gold. The electron-dense label absorbs electrons and can be visualized by the electron microscope.

4. Immunodetection Kits

In some aspects, the present embodiments concern immunodetection kits for use with the immunodetection methods described above. As anti-KD biomarker antibodies are generally used to detect such biomarker proteins, polypeptides and/or peptides, the antibodies will preferably be included in the kit. However, kits including both such components may be provided. Immunodetection kits will thus comprise, in suitable container means, a first antibody that binds to a biomarker protein, polypeptide and/or peptide (e.g., an anti-PDGFC antibody), and/or optionally, an immunodetection reagent and/or further optionally, a purified or recombinant biomarker protein, polypeptide and/or peptide.

In some embodiments, monoclonal antibodies will be used. In certain embodiments, the first antibody that binds to the biomarker protein, polypeptide and/or peptide may be pre-bound to a solid support, such as a column matrix and/or well of a microtitre plate.

Immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and/or all such labels may be employed in connection with the present embodiments.

Kits in accordance with the present embodiments may further comprise a suitably aliquoted composition of the biomarker protein, polypeptide and/or polypeptide, whether labeled and/or unlabeled, as may be used to prepare a standard curve for a detection assay. Provided kits may contain antibody-label conjugates either in fully conjugated faun, in the form of intermediates, and/or as separate moieties to be conjugated by the user of the kit. The components of the kits may be packaged either in aqueous media and/or in lyophilized form.

The container means of the kits will generally include at least one vial, test tube, flask, bottle, syringe and/or other container means, into which the antibody may be placed, and/or preferably, suitably aliquoted. The kits of the present embodiments will also typically include a means for containing the antibody, antigen, and/or any other reagent containers in close confinement for commercial sale. Such containers may include injection and/or blow-molded plastic containers into which the desired vials are retained.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification of KD Biomarkers

### Sample Collect and Processing

The study was approved by the institutional review board of Baylor Research Institute. Informed consents were obtained from all patients and healthy donors. Blood was collected into Tempus™ tubes (Applied Biosystems, Carlsbad, Calif.) or PaxGene tubes (Qiagen, Valencia, Calif.) from patients and healthy controls and delivered to Baylor Institute for Immunology Research and stored at −20° C. A summary of the patients used for the KD blood samples is provided as Table 1.

TABLE 1
KD patient samples

| Kawasaki Disease | Number |
|---|---|
| Total Patients | 98 |
| Patients with Pre and 24 h Post IVIG | 47 |
| Patients with Pre and 2 w or 4 w post | 13 |
| Patients with Pre and 1 y post | 1 |
| Patients with Pre only | 15 |
| Patients with 24 h post (no Pre) | 13 |
| Patients with 5 w post (no Pre) | 2 |
| Patients with unknown condition | 7 |

Total RNA was isolated from the whole blood lysate using MagMax™ total RNA extraction kit (Applied Biosystems, Carlsbad, Calif.), and globin mRNA was removed with GLOBINclear™ Whole Blood Globin Reduction Kit (Applied Biosystems, Carlsbad, Calif.). Agilent 2100 Bioanalyzer (Agilent, Palo Alto, Calif.) was used to measure RNA integrity number (RIN). Globin-reduced RNA with RIN>6 was further amplified and labeled with Illumina® TotalPrep™ RNA Amplification Kit (Applied Biosystems, Carlsbad, Calif.). cRNA was hybridized Human HT12 BeadChip array (Illumina®, San Diego, Calif.) and scanned on an Illumina® BeadStation 500. Fluorescent hybridization signals were assessed with GenomeStudio® software (Illumina®, San Diego, Calif.).

### Microarray Analysis

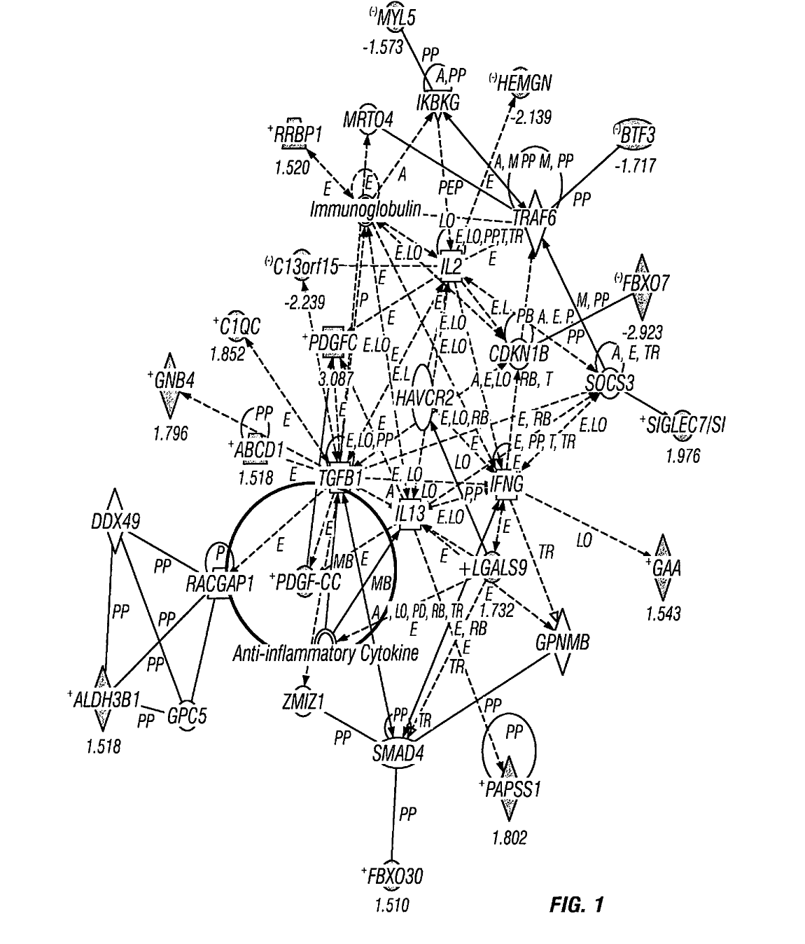

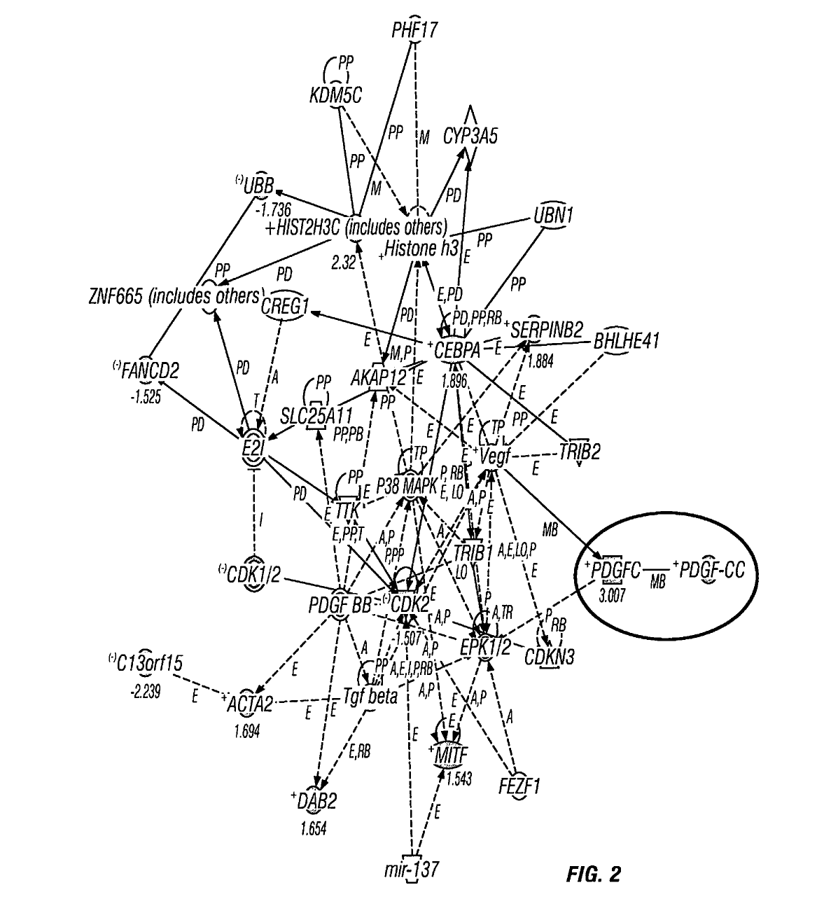

After background subtraction and average normalization, microarray data was analyzed using GeneSpring® 11.5 software (Agilent, Santa Clara, Calif.). Before analysis, the probes that were not expressed in any one of the samples were filtered out. Statistical analysis (Mann-Whitney U test with Benjamini-Hochberg Multiple testing correction) and fold change analysis were performed between disease group and its corresponding healthy control group. Analysis of Significance was performed by obtaining probes that were significant in Kawasaki Disease (P<0.05, Mann-Whitney U test with Benjamini-Hochberg Multiple testing correction, Fold change>1.5), but not in NOMID and SOJIA group (P>0.5), comparing with each dataset's own healthy controls. IPA software (Ingenuity System Redwood City, Calif.) was used to perform pathway analysis. For modular analysis, a set of 260 transcriptional modules were used as a pre-existing framework for the analysis. The approach used for the construction of such framework was previously reported (Chaussabel et al., 2008). Briefly, genes with coordinate expression within or across nine whole blood disease datasets where selected in multiple rounds of clique and paraclique clustering to form a 260 transcriptional module framework, and within each module, percentage of significant probes was assessed by T-test. Examples of signaling pathways with genes differentially regulated in KD are shown in FIGS. 1-2.

### RT-PCR

cDNA was generated from total mRNA using High Capacity Reverse Transcription kit (Applied Biosystems, Carlsbad, Calif.). Quantitative real-time PCR was performed using the TaqMan® Gene expression Assays on the LightCycler® 480 (Roche Applied Science, Indianapolis, Ind.) in 10 μl reaction volume. Taqman® Assay IDs for Human PDGFC gene are Hs00211916_ml, Hs01053574_ml and Hs01044216_ml (see Table 2 below). Threshold cycle (CT) values for PDGFC gene were normalized to the average of endogenous control genes phosphoglycerate kinase 1 (PGK1; NCBI accession no. NM_{—}000291.3) and TATA box binding protein (TBP; NCBI accession no. NM_{—}003194.4).

TABLE 2
PDGFC regions analyzed

| Taqman ® | Amplicon | mRNA | |
|---|---|---|---|
| Assay ID | length | detected | PDGFC encoded |
| Hs00211916_ml | 90 nucleotides | NR_036641.1; | Functional PDGFC ORF |
| | | NM_016205.2 | and nonsense transcript |
| Hs01053574_ml | 94 nucleotides | NR_036641.1 | Nonsense transcript |
| Hs01044216_ml | 98 nucleotides | NM_016205.2 | Functional PDGFC ORF |

### Results

More than 1700 transcripts were found to be differentially expressed in ex vivo blood samples from KD patients compared to healthy matched controls. KD patients also showed a down-regulation of transcripts associated with adaptive immunity and profound up-regulation in transcripts associated with inflammation relative to Systemic Lupus Erythematosus (SLE) patients. The KD-specific transcription profile was especially evident when compared to transcript expression in patients with other conditions similar to KD using an analysis of significance strategy. Thus, both neonatal onset multisystem inflammatory disease (NOMID) and systemic onset juvenile idiopathic arthritis (SoJIA), which are IL-1-mediated diseases that present with inflammation and system tissue damage, could be differentiated from KD by differential transcript expression using this analysis method (described in Allantaz et al., 2007, incorporated herein by reference).

Blood samples from KD patients showed decreased expression of transcripts from the LOC641518, C21orf57, UBB, FBXO7, LOC731777, BTF3, C13orf15, SFRS2B, HEMGN, HPS1, IFT52, FAM10A7, IFT52, LOC441714, IMMP2L, TMEM57, IFRD2, LOC646784, PYROXD1, MIR155HG, ZNF138, TCC39B, OR7E156P, FANCD2, XPOT, AZIN1, BLOC152, CDK2, MYL5, HRASLS2 and TMCC1 genes. On the other hand, KD blood samples were found to have increased expression of transcripts from the EPSTI1, OASL, CEBPA, C9orf167, FHOD1, ALDH3B1, LRSAM1, SIGLEC7, SLC24A4, GAA, RRBP1, DAB2, HIST2H3C, LGALS9, GPR177, CMTM4, FBXO30, WSB2, PAPSS1, SERPINB2, ACTA2, LOC729417, ABCD1, GNB4, MITF, C1QC, CCDC24, PGM5, LOC729816, OLFM4 and PDGFC genes. In particular, both microarray and RT-PCR demonstrated that PDGFC mRNA levels were significantly elevated in KD patients compared to healthy children and with children suffering from other inflammatory diseases. KD specific transcripts were then analyzed for their roles in signaling pathways involved in inflammation (FIG. 1) and connective tissue development (FIG. 2) to determine which of the markers might have a primary role in the disease.

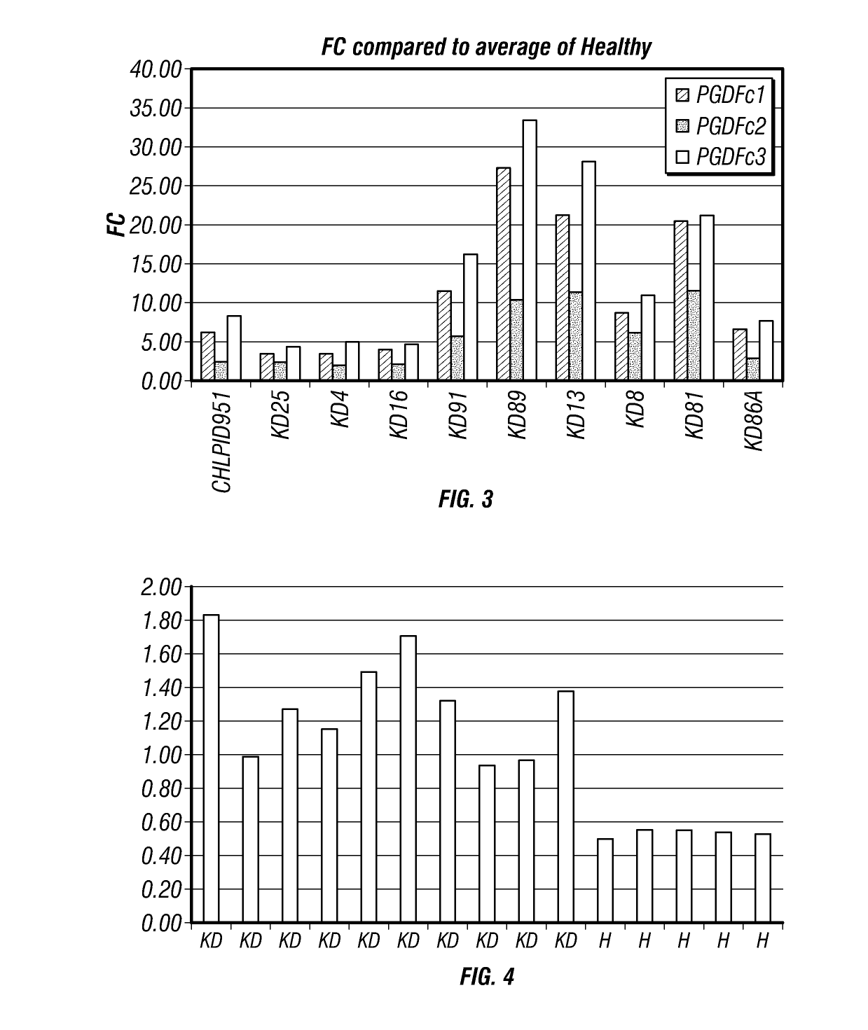

From these analyses PDGFC was also indicated as a primary actor in KD, and was thus subjected to further study. Quantitative RT-PCR demonstrated that PDGFC transcripts were up-regulated 5- to 30-fold in KD patients (FIG. 3). This elevated expression was evident using primer pairs that amplified three different regions of PDGFC transcript. Importantly, the elevated expression was most apparent in primer pairs that amplified transcripts that encoded functional PDGFC protein. A further comparison of the expression of the PDGFC transcripts encoding functional versus nonfunctional PDGFC protein indicated that KD patients preferentially express functional PDGFC transcripts (FIG. 4).

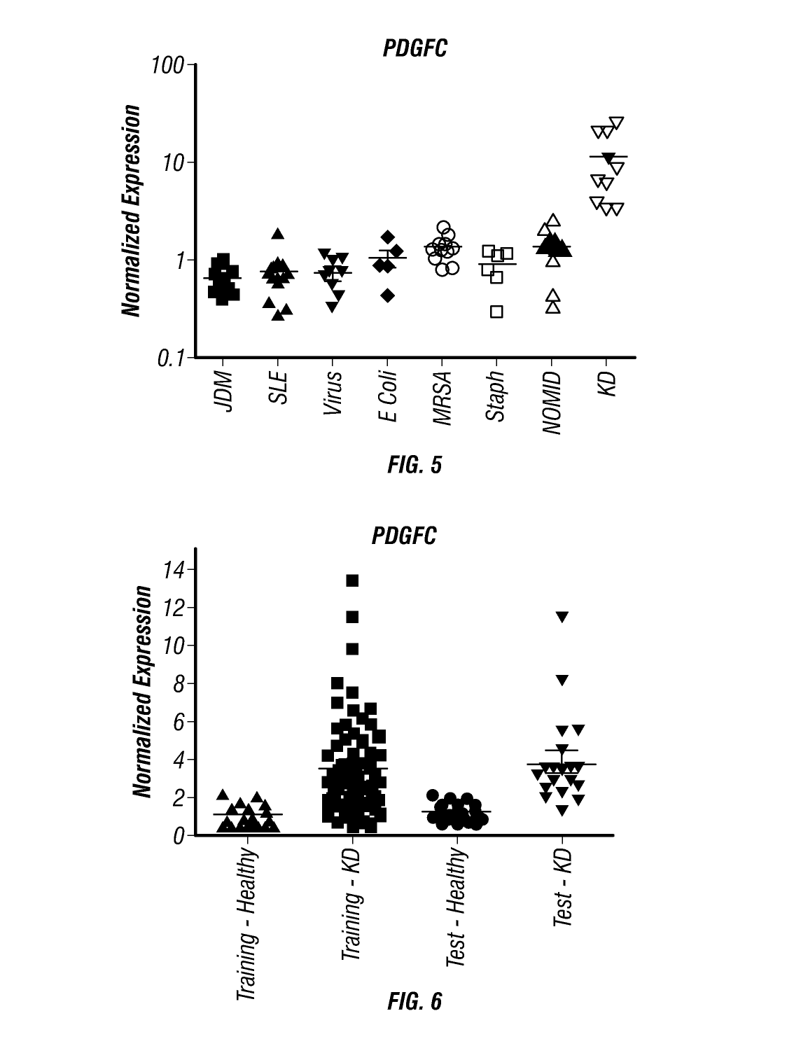

PDGFC transcript levels in whole blood from KD and other febrile diseases were evaluated with quantitative RT-PCR (ragman assay Hs00211916_ml) (FIG. 5). Expression values of PDGFC were not changed significantly in patients with Juvenile dermatomyositis (JDM), Systemic lupus erythematosus (SLE), Rhinovirus, E. coli, Methicillin-resistant Staphylococcus aureus (MRSA), staphylococcus aureus (Staph) and Neonatal onset multisystem inflammatory disease (NOMID) (FIG. 5). The elevated expression of PDGFC was found in KD patients with 5 to 30 fold increase (FIG. 5).

Microarray analysis suggested PDGFC transcription is up-regulated in KD patients in two independent cohorts of KD samples (n=66 and n=19) (FIG. 6).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 5,279,721
U.S. Patent Publn. 20090304680
U.S. Patent Publn. 20110189698
Abbondanzo et al., Breast Cancer Res. Treat., 16:182(151), 1990.
Allantaz et al., J. Exp. Med., 204(9):2131-2144, 2007.
Allred et al., Breast Cancer Res. Treat., 16:182(149), 1990.
Brown et al. Immunol. Ser., 53:69-82, 1990.
Chaussabel et al., Immunity, 29(1):150-164, 2008.
De Jager et al., Semin. Nucl. Med., 23(2):165-179, 1993.
Doolittle and Ben-Zeev, Methods Mol. Biol., 109:215-237, 1999.
Gulbis and Galand, Hum. Pathol. 24(12):1271-1285, 1993.
Nakamura et al., In: Enzyme Immunoassays: Heterogeneous and Homogeneous Systems, Chapter 27, 1987.
Reigstad et al., FEBS J., 272:5723-5741, 2005.

What is claimed is:

1. A method for detecting a biomarker of Kawasaki disease (KD) in a subject comprising determining a platelet-derived growth factor C (PDGFC) expression level in a biological sample from a subject suspected of having or at risk for having KD, wherein elevated PDGFC expression relative to a reference level identifies the subject as having a biomarker of KD.

2. The method of claim 1, wherein the biological sample is a serum sample.

3. The method of claim 1, wherein the subject suspected of having or at risk for having KD exhibits one or more of the following symptoms: oral erythema; rash; swollen lips; cracked lips; swelling of the hands; swelling of the feet; eye redness; uveitis; aseptic meningitis; lymph node inflammation; vascular inflammation; coronary aneurism; fever; joint pain; joint swelling; or peeling skin over nail beds, palms, soles and groin area.

4. The method of claim 1, wherein the subject suspected of having or at risk for having KD is of Asian ancestry.

5. The method of claim 1, further comprising obtaining the biological sample from the subject.

6. The method of claim 1, wherein the PDGFC expression level is determined by determining PDGFC RNA expression in the sample.

7. The method of claim 6, wherein determining PDGFC RNA expression comprises determining expression of PDGFC RNA encoding an active polypeptide.

8. The method of claim 6, wherein determining PDGFC RNA expression comprises nucleic acid hybridization or nucleic acid sequencing.

9. The method of claim 6, wherein determining PDGFC RNA expression comprises RT-PCR.

10. The method of claim 6, wherein the elevated PDGFC expression is between about 3- and about 50-fold greater PDGFC RNA expression relative to the reference level.

11. The method of claim 1, wherein the reference level represents a PDGFC expression level from a subject who does not have KD.

12. The method of claim 1, further comprising determining the expression level of at least second gene in the sample.

13. The method of claim 12, wherein the second gene is a control gene.

14. The method of claim 12, wherein the second gene is selected from the group consisting of LOC641518, C21orf57, UBB, FBXO7, LOC731777, BTF3, C13orf15, SFRS2B, HEMGN, HPS1, IFT52, FAM10A7, IFT52, LOC441714, IMMP2L, TMEM57, IFRD2, LOC646784, PYROXD1, MIR155HG, ZNF138, TCC39B, OR7E156P, FANCD2, XPOT, AZIN1, BLOC152, CDK2, MYL5, HRASLS2, TMCC1, EPSTI1, OASL, CEBPA, C9orf167, FHOD1, ALDH3B1, LRSAM1, SIGLEC7, SLC24A4, GAA, RRBP1, DAB2, HIST2H3C, LGALS9, GPR177, CMTM4, FBXO30, WSB2, PAPSS1, SERPINB2, ACTA2, LOC729417, ABCD1, GNB4, MITF, C1QC, CCDC24, PGM5, LOC729816 and OLFM4.

15. The method of claim 1, further comprising reporting the PDGFC expression in the sample.

16. The method of claim 15, wherein the PDGFC expression is reported in a written report.

17. The method of claim 1, further comprising determining whether the subject has a biomarker of KD by comparing the PDGFC expression level in the sample to a reference level.

18. The method of claim 17, further comprising reporting whether the subject has a biomarker of KD.

19. The method of claim 1, wherein the subject does not have an elevated level of PDGFC expression.

20. A method for treating a subject with Kawasaki disease (KD) comprising:
(a) evaluating expression of platelet-derived growth factor C (PDGFC) in the subject; and
(b) administering an anti-KD therapy to the subject if the subject exhibits elevated PDGFC expression relative to a reference level.

21. The method of claim 20, wherein the anti-KD therapy comprises administration IgG.

22. The method of claim 20, wherein the anti-KD therapy comprises administration of aspirin, corticosteroids or an anti-TNFα therapy.

23. The method of claim 20, wherein an anti-KD therapy was administered to the subject before said evaluating.

24. The method of claim 20, wherein the subject exhibits one or more of the following symptoms: oral erythema; rash; swollen lips; cracked lips; swelling of the hands; swelling of the feet; eye redness; uveitis; aseptic meningitis; lymph node inflammation; vascular inflammation; coronary aneurism; fever; joint pain; joint swelling; or peeling skin over nail beds, palms, soles and groin area.

25. The method of claim 20, wherein the PDGFC expression is PDGFC RNA expression.

26. The method of claim 25, wherein the PDGFC RNA expression is expression of PDGFC RNA encoding an active polypeptide.

27. The method of claim 20, wherein evaluating expression of PDGFC comprises measuring PDGFC expression.

28. The method of claim 25, wherein the elevated PDGFC expression is between about 3- and about 50-fold greater PDGFC RNA expression relative to the reference level.

29. The method of claim 20, wherein the reference level represents a PDGFC expression level from a subject who does not have KD.

30. The method of claim 20, further comprising evaluating the expression level of at least a second gene in the subject.

31. The method of claim 30, wherein evaluating the expression of at least a second gene comprises measuring at least a second gene expression.

32. The method of claim 30, wherein the second gene is a control gene.

33. The method of claim 30, wherein the second gene is selected from the group consisting of LOC641518, C21orf57, UBB, FBXO7, LOC731777, BTF3, C13orf15, SFRS2B, HEMGN, HPS1, IFT52, FAM10A7, IFT52, LOC441714, IMMP2L, TMEM57, IFRD2, LOC646784, PYROXD1, MIR155HG, ZNF138, TCC39B, OR7E156P, FANCD2, XPOT, AZIN1, BLOC152, CDK2, MYL5, HRASLS2, TMCC1, EPSTI1, OASL, CEBPA, C9orf167, FHOD1, ALDH3B1, LRSAM1, SIGLEC7, SLC24A4, GAA, RRBP1, DAB2, HIST2H3C, LGALS9, GPR177, CMTM4, FBXO30, WSB2, PAPSS1, SERPINB2, ACTA2, LOC729417, ABCD1, GNB4, MITF, C1QC, CCDC24, PGM5, LOC729816 and OLFM4.

34. A method for treating a subject with Kawasaki disease (KD) comprising:
(a) administering an anti-KD therapy to the subject;
(b) evaluating expression of platelet-derived growth factor C (PDGFC) in the subject; and
(c) administering a further anti-KD therapy to the subject if the subject exhibits elevated PDGFC expression relative to a reference level.

35. A method for treating Kawasaki disease (KD) comprising administering an anti-KD therapy to a subject determined to have elevated platelet-derived growth factor C (PDGFC) expression relative to a reference level.

36. The method of claim 35, wherein the anti-KD therapy comprises administration IgG.

37. The method of claim 35, wherein the anti-KD therapy comprises administration of aspirin, corticosteroids or an anti-TNFα therapy.

38. The method of claim 35, wherein the subject exhibits one or more of the following symptoms: oral erythema; rash; swollen lips; cracked lips; swelling of the hands; swelling of the feet; eye redness; uveitis; aseptic meningitis; lymph node inflammation; vascular inflammation; coronary aneurism; fever; joint pain; joint swelling; or peeling skin over nail beds, palms, soles and groin area.

39. The method of claim 35, wherein the elevated PDGFC expression level is an elevated PDGFC RNA expression level.

40. The method of claim 39, wherein the elevated PDGFC RNA expression is elevated expression of PDGFC RNA encoding an active polypeptide.

41. The method of claim 39, wherein the elevated PDGFC expression is about is between about 3- and about 50-fold greater PDGFC RNA expression relative to the reference level.

42. The method of claim 35, wherein the reference level represents a PDGFC expression level from a subject who does not have KD.

43. The method of claim 35, wherein the subject is determined to have elevated EPSTI1, OASL, CEBPA, C9orf167, FHOD1, ALDH3B1, LRSAM1, SIGLEC7, SLC24A4, GAA, RRBP1, DAB2, HIST2H3C, LGALS9, GPR177, CMTM4, FBXO30, WSB2, PAPSS1, SERPINB2, ACTA2, LOC729417, ABCD1, GNB4, MITF, C1QC, CCDC24, PGM5, LOC729816 or OLFM4 expression relative to a reference level.

44. The method of claim 35, wherein the subject is determined to have decreased LOC641518, C21orf57, UBB, FBXO7, LOC731777, BTF3, C13orf15, SFRS2B, HEMGN, HPS1, IFT52, FAM10A7, IFT52, LOC441714, IMMP2L, TMEM57, IFRD2, LOC646784, PYROXD1, MIR155HG, ZNF138, TCC39B, OR7E156P, FANCD2, XPOT, AZIN1, BLOC152, CDK2, MYL5, HRASLS2 or TMCC1 expression relative to a reference level.

45. A tangible computer-readable medium comprising computer-readable code that, when executed by a computer, causes the computer to perform operations comprising:
a) receiving information corresponding to a level of expression of platelet-derived growth factor C (PDGFC) in a sample from a subject suspected of having or at risk for having KD; and
b) determining a relative level of expression of PDGFC compared to a reference level, wherein elevated PDGFC expression relative to the reference level indicates the presence of a biomarker of KD.

46. The method of claim 45, further comprising receiving information corresponding to a reference level of expression of PDGFC in a sample from a healthy subject.

47. The method of claim 45, wherein the reference level is stored in said tangible computer-readable medium.

48. The tangible computer-readable medium of claim 45, wherein the receiving information comprises receiving from a tangible data storage device information corresponding to a level of expression of PDGFC in a sample from a subject suspected of having or at risk for having KD.

49. The tangible computer-readable medium of claim 45, further comprising computer-readable code that, when executed by a computer, causes the computer to perform one or more additional operations comprising: sending information corresponding to the relative level of expression of PDGFC to a tangible data storage device.

50. The tangible computer-readable medium of claim 45, wherein the receiving information further comprises receiving information corresponding to a level of expression of at one of LOC641518, C21orf57, UBB, FBXO7, LOC731777, BTF3, C13orf15, SFRS2B, HEMGN, HPS1, IFT52, FAM10A7, IFT52, LOC441714, IMMP2L, TMEM57, IFRD2, LOC646784, PYROXD1, MIR155HG, ZNF138, TCC39B, OR7E156P, FANCD2, XPOT, AZIN1, BLOC152, CDK2, MYL5, HRASLS2, TMCC1, EPSTI1, OASL, CEBPA, C9orf167, FHOD1, ALDH3B1, LRSAM1, SIGLEC7, SLC24A4, GAA, RRBP1, DAB2, HIST2H3C, LGALS9, GPR177, CMTM4, FBXO30, WSB2, PAPSS1, SERPINB2, ACTA2, LOC729417, ABCD1, GNB4, MITF, C1QC, CCDC24, PGM5, LOC729816 or OLFM4 in a sample from a subject suspected of having or at risk for having KD.

51. The tangible computer-readable medium of claim 45, wherein the computer-readable code that, when executed by a computer, causes the computer to perform operations further comprising: c) calculating a diagnostic score for the sample, wherein the diagnostic score is indicative of the probability that the sample is from a subject having KD.


SEQUENCE LISTING (3 sequences)

SEQ ID NO: 1 - RNA, 3018 nt, Homo sapiens
gcccggagagccgcaucuauuggcagcuuuguuauugaucagaaacugcucgccgccgacuuggcuuccagucuggcugcgggcaacccuugaguuuucgccucuguccugucccccgaacugacaggugcucccagcaacuugcuggggacuucucgccgcucccccgcguccccacccccucauuccucccucgccuucacccccacccccaccacuucgccacagcucaggauuuguuuaaaccuugggaaacugguucagguccagguuuugcuuugauccuuuucaaaaacuggagacacagaagagggcucuaggaaaaaguuuuggaugggauuauguggaaacuacccugcgauucucugcugccagagcaggcucggcgcuuccaccccagugcagccuuccccuggcgguggugaaagagacucgggagucgcugcuuccaaagugcccgccgugagugagcucucaccccagucagccaaaugagccucuucgggcuucuccugcugacaucugcccuggccggccagagacaggggacucaggcggaauccaaccugaguaguaaauuccaguuuuccagcaacaaggaacagaacggaguacaagauccucagcaugagagaauuauuacugugucuacuaauggaaguauucacagcccaagguuuccucauacuuauccaagaaauacggucuugguauggagauuaguagcaguagaggaaaauguauggauacaacuuacguuugaugaaagauuugggcuugaagacccagaagaugacauaugcaaguaugauuuuguagaaguugaggaacccagugauggaacuauauuagggcgcuggugugguucugguacuguaccaggaaaacagauuucuaaaggaaaucaaauuaggauaagauuuguaucugaugaauauuuuccuucugaaccaggguucugcauccacuacaacauugucaugccacaauucacagaagcugugaguccuucagugcuacccccuucagcuuugccacuggaccugcuuaauaaugcuauaacugccuuuaguaccuuggaagaccuuauucgauaucuugaaccagagagauggcaguuggacuuagaagaucuauauaggccaacuuggcaacuucuuggcaaggcuuuuguuuuuggaagaaaauccagagugguggaucugaaccuucuaacagaggagguaagauuauacagcugcacaccucguaacuucucaguguccauaagggaagaacuaaagagaaccgauaccauuuucuggccagguugucuccugguuaaacgcuguggugggaacugugccuguugucuccacaauugcaaugaaugucaaugugucccaagcaaaguuacuaaaaaauaccacgagguccuucaguugagaccaaagaccggugucaggggauugcacaaaucacucaccgacguggcccuggagcaccaugaggagugugacugugugugcagagggagcacaggaggauagccgcaucaccaccagcagcucuugcccagagcugugcagugcaguggcugauucuauuagagaacguaugcguuaucuccauccuuaaucucaguuguuugcuucaaggaccuuucaucuucaggauuuacagugcauucugaaagaggagacaucaaacagaauuaggaguugugcaacagcucuuuugagaggaggccuaaaggacaggagaaaaggucuucaaucguggaaagaaaauuaaauguuguauuaaauagaucaccagcuaguuucagaguuaccauguacguauuccacuagcuggguucuguauuucaguucuuucgauacggcuuaggguaaugucaguacaggaaaaaaacugugcaagugagcaccugauuccguugccuugcuuaacucuaaagcuccauguccugggccuaaaaucguauaaaaucuggauuuuuuuuuuuuuuuuugcucauauucacauauguaaaccagaacauucuauguacuacaaaccugguuuuuaaaaaggaacuauguugcuaugaauuaaacuugugucgugcugauaggacagacuggauuuuucauauuucuuauuaaaauuucugccauuuagaagaagagaacuacauucaugguuuggaagagauaaaccugaaaagaagaguggccuuaucuucacuuuaucgauaagucaguuuauuuguuucauuguguacauuuuuauauucuccuuuugacauuauaacuguuggcuuuucuaaucuuguuaaauauaucuauuuuuaccaaagguauuuaauauucuuuuuuaugacaacuuagaucaacuauuuuuagcuugguaaauuuuucuaaacacaauuguuauagccagaggaacaaagaugauauaaaauauuguugcucugacaaaaauacauguauuucauucucguauggugcuagaguuagauuaaucugcauuuuaaaaaacugaauuggaauagaauugguaaguugcaaagacuuuuugaaaauaauuaaauuaucauaucuuccauuccuguuauuggagaugaaaauaaaaagcaacuuaugaaaguagacauucagauccagccauuacuaaccuauuccuuuuuuggggaaaucugagccuagcucagaaaaacauaaagcaccuugaaaaagacuuggcagcuuccugauaaagcgugcugugcugugcaguaggaacacauccuauuuauugugauguugugguuuuauuaucuuaaacucuguuccauacacuuguauaaauacauggauauuuuuauguacagaaguaugucucuuaaccaguucacuuauuguacucuggcaauuuaaaagaaaaucaguaaaauauuuugcuuguaaaaugcuuaauaucgugccuagguuauguggugacuauuugaaucaaaaauguauugaaucaucaaauaaaagaauguggcuauuuuggggagaaaauuaaaaaaaaaaa

SEQ ID NO: 2 - protein, 345 aa, Homo sapiens
MSLFGLLLLTSALAGQRQGTQAESNLSSKFQFSSNKEQNGVQDPQHERIITVSTNGSIHSPRFPHTYPRNTVLVWRLVAVEENVWIQLTFDERFGLEDPEDDICKYDFVEVEEPSDGTILGRWCGSGTVPGKQISKGNQIRIRFVSDEYFPSEPGFCIHYNIVMPQFTEAVSPSVLPPSALPLDLLNNAITAFSTLEDLIRYLEPERWQLDLEDLYRPTWQLLGKAFVFGRKSRVVDLNLLTEEVRLYSCTPRNFSVSIREELKRTDTIFWPGCLLVKRCGGNCACCLHNCNECQCVPSKVTKKYHEVLQLRPKTGVRGLHKSLTDVALEHHEECDCVCRGSTGG

SEQ ID NO: 3 - RNA, 3079 nt, Homo sapiens
gcccggagagccgcaucuauuggcagcuuuguuauugaucagaaacugcucgccgccgacuuggcuuccagucuggcugcgggcaacccuugaguuuucgccucuguccugucccccgaacugacaggugcucccagcaacuugcuggggacuucucgccgcucccccgcguccccacccccucauuccucccucgccuucacccccacccccaccacuucgccacagcucaggauuuguuuaaaccuugggaaacugguucagguccagguuuugcuuugauccuuuucaaaaacuggagacacagaagagggcucuaggaaaaaguuuuggaugggauuauguggaaacuacccugcgauucucugcugccagagcaggcucggcgcuuccaccccagugcagccuuccccuggcgguggugaaagagacucgggagucgcugcuuccaaagugcccgccgugagugagcucucaccccagucagccaaaugagccucuucgggcuucuccugcugacaucugcccuggccggccagagacaggggacucaggcggaauccaaccugaguaguaaauuccaguuuuccagcaacaaggaacagaacgguaggaacuauauccaagcaucuggacuggcauagaaaagaggagaaagaacauuuaaaaggaguacaagauccucagcaugagagaauuauuacugugucuacuaauggaaguauucacagcccaagguuuccucauacuuauccaagaaauacggucuugguauggagauuaguagcaguagaggaaaauguauggauacaacuuacguuugaugaaagauuugggcuugaagacccagaagaugacauaugcaaguaugauuuuguagaaguugaggaacccagugauggaacuauauuagggcgcuggugugguucugguacuguaccaggaaaacagauuucuaaaggaaaucaaauuaggauaagauuuguaucugaugaauauuuuccuucugaaccaggguucugcauccacuacaacauugucaugccacaauucacagaagcugugaguccuucagugcuacccccuucagcuuugccacuggaccugcuuaauaaugcuauaacugccuuuaguaccuuggaagaccuuauucgauaucuugaaccagagagauggcaguuggacuuagaagaucuauauaggccaacuuggcaacuucuuggcaaggcuuuuguuuuuggaagaaaauccagagugguggaucugaaccuucuaacagaggagguaagauuauacagcugcacaccucguaacuucucaguguccauaagggaagaacuaaagagaaccgauaccauuuucuggccagguugucuccugguuaaacgcuguggugggaacugugccuguugucuccacaauugcaaugaaugucaaugugucccaagcaaaguuacuaaaaaauaccacgagguccuucaguugagaccaaagaccggugucaggggauugcacaaaucacucaccgacguggcccuggagcaccaugaggagugugacugugugugcagagggagcacaggaggauagccgcaucaccaccagcagcucuugcccagagcugugcagugcaguggcugauucuauuagagaacguaugcguuaucuccauccuuaaucucaguuguuugcuucaaggaccuuucaucuucaggauuuacagugcauucugaaagaggagacaucaaacagaauuaggaguugugcaacagcucuuuugagaggaggccuaaaggacaggagaaaaggucuucaaucguggaaagaaaauuaaauguuguauuaaauagaucaccagcuaguuucagaguuaccauguacguauuccacuagcuggguucuguauuucaguucuuucgauacggcuuaggguaaugucaguacaggaaaaaaacugugcaagugagcaccugauuccguugccuugcuuaacucuaaagcuccauguccugggccuaaaaucguauaaaaucuggauuuuuuuuuuuuuuuuugcucauauucacauauguaaaccagaacauucuauguacuacaaaccugguuuuuaaaaaggaacuauguugcuaugaauuaaacuugugucgugcugauaggacagacuggauuuuucauauuucuuauuaaaauuucugccauuuagaagaagagaacuacauucaugguuuggaagagauaaaccugaaaagaagaguggccuuaucuucacuuuaucgauaagucaguuuauuuguuucauuguguacauuuuuauauucuccuuuugacauuauaacuguuggcuuuucuaaucuuguuaaauauaucuauuuuuaccaaagguauuuaauauucuuuuuuaugacaacuuagaucaacuauuuuuagcuugguaaauuuuucuaaacacaauuguuauagccagaggaacaaagaugauauaaaauauuguugcucugacaaaaauacauguauuucauucucguauggugcuagaguuagauuaaucugcauuuuaaaaaacugaauuggaauagaauugguaaguugcaaagacuuuuugaaaauaauuaaauuaucauaucuuccauuccuguuauuggagaugaaaauaaaaagcaacuuaugaaaguagacauucagauccagccauuacuaaccuauuccuuuuuuggggaaaucugagccuagcucagaaaaacauaaagcaccuugaaaaagacuuggcagcuuccugauaaagcgugcugugcugugcaguaggaacacauccuauuuauugugauguugugguuuuauuaucuuaaacucuguuccauacacuuguauaaauacauggauauuuuuauguacagaaguaugucucuuaaccaguucacuuauuguacucuggcaauuuaaaagaaaaucaguaaaauauuuugcuuguaaaaugcuuaauaucgugccuagguuauguggugacuauuugaaucaaaaauguauugaaucaucaaauaaaagaauguggcuauuuuggggagaaaauuaaaaaaaaaaa